(19) United States
(12) Plant Patent Application Publication
(10) Pub. No.: US 2020/0113097 P1
(43) Pub. Date: Apr. 9, 2020

(54) Turpentine Bush Plant Named 'Butterbloom'

(50) ;

(71) Applicant: Nicholas Benoit Shipley, Tucson, AZ (US)

(72) Inventor: Nicholas Benoit Shipley, Tucson, AZ (US)

(21) Appl. No.: 16/350,154

(22) Filed: Oct. 9, 2018

(51) Int. Cl. A01H 6/36

(52) U.S. Cl. PLT/226

(57) ABSTRACT

A new and distinct Ericameria laricifolia plant named Butterbloom is characterized by compact habit, even growth and excellent tolerance to pruning.

Latin name: Ericameria laricifolia.

Varietal denomination: ‘Butterbloom’.

### BACKGROUND OF THE INVENTION

Turpentine bush, Ericameria laricifolia is an evergreen, much branched, bright green shrub with soft, needle like resinous leaves native to much of southwestern North America between the elevations of about 2500-6000 feet. Ericameria laricifolia is a member of the Ericameria section of the genus Ericameria which is a member of the Astereae tribe of the sunflower family, Asteraceae. Ericameria laricifolia can be found in the states of California, Nevada, Utah, Arizona, New Mexico, and Texas in the U.S. and in the states of Sonora and Chihuahua, Mexico. Turpentine bush illuminates the hillsides and mountains of the Southwest with its bright yellow flowers from August-November, although some flowers can be produced nearly year round under favorable conditions. Plants in the wild vary in size from as small as 1×1 foot (height×width) to as large as 4×6 feet depending upon site growth conditions and population variation. Turpentine bush is a popular landscape plant in the southwestern U.S. finding uses as a foundation plant, low informal or clipped hedge, seasonal color plant as well as a specimen shrub. In cultivation most cultivars will reach larger maximum size listed above without pruning. Ericameria laricifolia generally tolerates pruning well and is long lived in the landscape. The present invention relates to a new and distinct cultivar of Ericameria laricifolia. The cultivar originated in seed growout in 2008 at a plant nursery near Tucson, Ariz. and is the object of this application.

### SUMMARY OF THE INVENTION

Among the features that distinguish the new turpentine bush cultivar from all other available and commercial varieties of turpentine bush known to the inventor are the following combination of characteristics: plants of even and compact growth form, as if hedged as well as very tolerant of pruning. Irrigated plants at 4 years of age are about 3 feet tall by 5 feet wide.

The propagation procedure is as follows: Cuttings are taken from long sprouts with growth less than one year old. These sprouts are cut into 3 inch long cuttings, then placed in DIP'N GRO™ solution (20 parts water to 1 part DIP'N GRO™) for 15 seconds, then planted into a tray filled with a 50% peat/50% perlite mix. Trays are placed into a rooting greenhouse maintained between 70-85° F. and relative humidity between 85-93% under mist watering. Bottom heat is applied as needed during the cool season only. Rooting is generally completed within 3½ weeks. The foregoing characteristics and distinctions come true to form and are established and transmitted through succeeding propagations. The present invention has not been evaluated under all possible environmental conditions, such that the phenotype may vary with variations in environment without a change in the genotype of the plant.

### BRIEF DESCRIPTION OF THE DRAWINGS

The accompanying photographs illustrate Ericameria laricifolia ‘Butterbloom’ growing near Tucson, Ariz., depicted in color as nearly correct as it is possible to make in a color illustration of the character.

FIG. 1 shows Ericameria laricifolia ‘Butterbloom’ at age 3 in bloom near Tucson, Ariz.

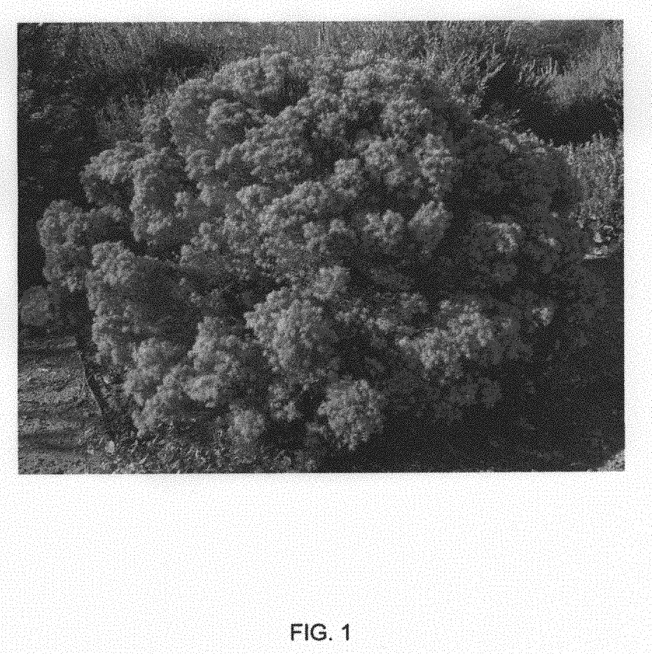

FIG. 2 shows Ericameria laricifolia ‘Butterbloom’ flowering heads at various stages of development.

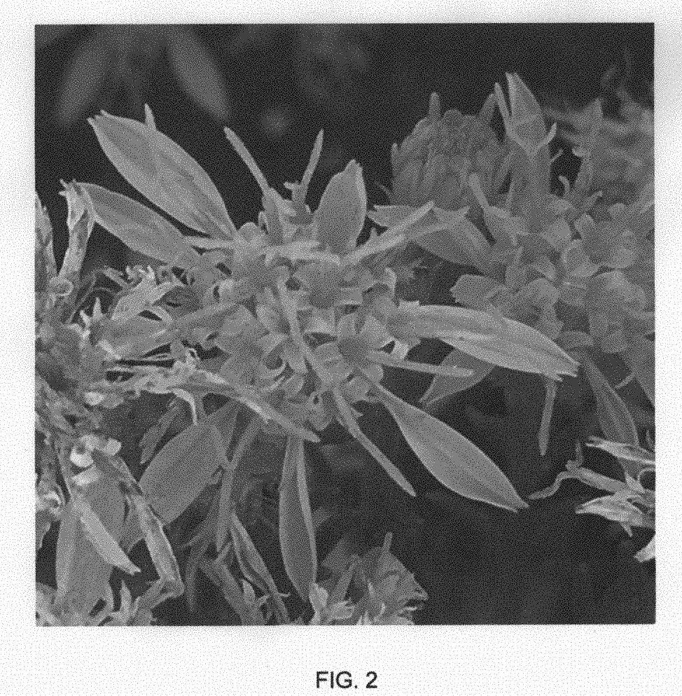

FIG. 3 shows Ericameria laricifolia ‘Butterbloom’ leaves, stems and an inflorescence in early bloom

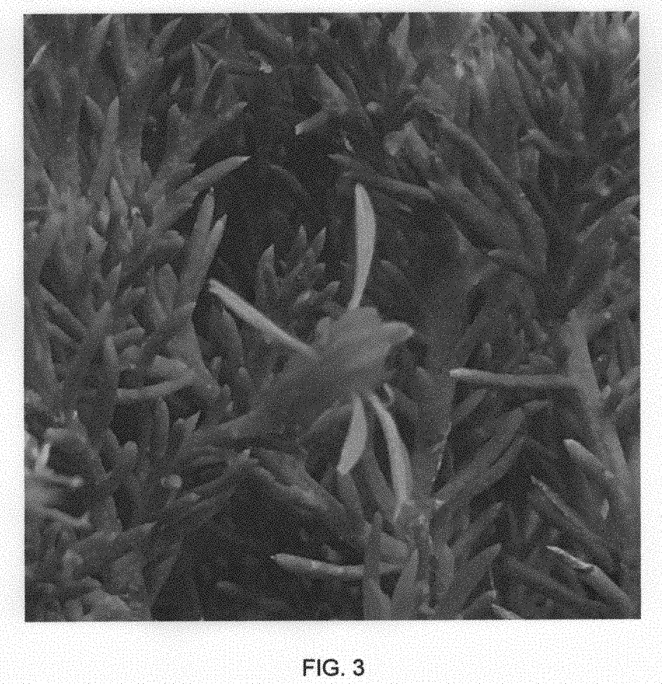

### DETAILED PLANT DESRIPTION

The following is a detailed description of the new turpentine bush plant based upon potted plants at about age 1 year and a more mature specimen growing near Tucson, Ariz. aged 4 years. The color descriptions are based upon The 5^{th} edition R.H.S. Colour Chart. Color names other than common usage are as listed in COLOR Universal Language and Dictionary of Names, by Kenneth L. Kelly and Deane B. Judd; National Bureau of Standards special publication 440. Washington, D.C.: U.S. Department of Commerce, National Bureau of Standards, December 1976.

Plant is a much branched evergreen shrub, reaching 3 feet tall×5 feet wide by age 4 years.
Leaves: Alternate, glabrous, resinous, containing numerous tiny glands (0.1 mm diameter, color closest to 141B), leaves highly variable in size and profile, shade leaves being quite flattened, while leaves grown in full sun appear needle like. Shade leaves 1.5-2 mm wide×11-24 mm long with a barely visible midrib, veins not visible. Shade leaves linear, apiculate, acute at the base, the leaf flattened, entire, color 141B. Sun leaves 1-1.25 mm wide×10-12 mm, approaching terete in profile except for some axial flattening. Midrib not visible. Leaves linear, apiculate, entire and acute at the base. Color closest to 141B. Stipules absent. Axillary buds obscure.
Stems: Highly branched, young stems similar to the leaves in color (141C), resinous surface and glands. Young stems faintly striate. Internode length 2-7 mm, stem diameter 0.75-1.5 mm. As stems mature the color grades to 164C at 2 mm diameter and 199D at 5 mm or more. At about 3 mm diameter the bark begins to shred longitudinally. This process continues as the stems thicken. By the time the stems reach 2-3 inches in diameter the bark becomes mottled and longitudinally furrowed and rough to the touch. Color grades from 164D to 201B on older bark. Flowers arranged in terminal heads, the heads measuring about 18 mm long×18-20 mm wide including the flowers. Involucre comprised of graduated phyllaries. Involucre measures 5-7 mm long×3-4 mm in diameter, more or less campanulate in shape.
Phyllaries: Somewhat spreading at the tips, glabrous, entire, apiculate, lanceolate. Each phyllary with a pair of glands at the base, these more or less spherical, 0.5 mm diameter, color 143B. Basal phyllaries 3 mm long×1 mm wide; apical phyllaries 5 mm long×1.5 mm wide. The longitudinal central ¼ portion of the phyllaries is colored 145A, which grades to scarious margins colored 150D. The tips of the apical phyllaries are colored 165A, while the tips of the basal phyllaries is colored as the central portion (145A).
Ray flowers: Numbering 6-8, typically 7, pistillate, lacking stamens. Ovary inferior, measuring 2-3 mm long×1 mm wide, colored 154A, oblanceoloid in shape, somewhat flattened dorsally, the surface sericeous. Corolla fused into a tube with a strap shaped radial extension. Corolla tube 3 mm long×0.5 mm diameter. Corolla strap 7 mm long×2 mm wide, elliptical in shape with 2-3 fine teeth at the apex. Corolla color 2A. Pappus of about 15 capillary bristles attached at the apex of the ovary, 4-6 mm long. Stigma exserted, 2 lobed, the recurved lobes colored 2A. Stigma lobes measure 2.5 mm long×0.2 mm diameter. Style about 6-7 mm long×0.25 mm diameter, colored like the corolla. Unopened mature buds oblanceoloid, 7 mm long×1 mm diameter (8 mm long including ovary) colored 2A at the apex grading to 1C near the base.
Disc flowers: Numbering 7-11 per head, perfect, protandrous. Corolla of 5 petals fused into a flaring tube with 5 acute radial petal lobes, these recurved and glabrous, the lobes measuring 2 mm long×1 mm wide. Corolla glabrous except for scattered short hairs below the Corolla lobes. Corolla translucent, color closest to 1C. Tube colored as corolla lobes. Corolla measures 6-7 mm long. Stamens 5, united into a tube around the style, inserted in the corolla. Stamens mature more or less at the time of ray flower opening but before the stigmas of the disc flowers have matured. Stamens at maturity are colored 13A, gradually changing to 22A as they age. Dry anthers measure 3 mm long×0.5 mm diameter. Pollen color 13A, released 1-2 days prior to petal spreading. Pappus of numerous capillary bristles (about 20) from 5-7 mm long, the pappus attached near the apex of the ovary. Ovary about 3 mm long×1 mm wide, oblanceoloid, sericeous, color close to 154A. Style+stigma length 10 mm long, 0.2-0.25 mm diameter. Style colored 1C. Stigma 2 lobed, each about 2-3 mm long, colored 18A, spreading, from slightly ascending to slightly descending. The terminal portion of the stigma lobes is covered with fine, spreading hairs. Unopened mature disc flower buds oblanceoloid, 11 mm long to top of ovary, 1 mm in diameter, color 13C.
Fruit: An achene 3.5-4 mm long, 0.75 mm thick at the widest point, dorsally compressed, angular, sericeous. Wind dispersed by the still attached pappus, the longest bristles 6 mm long. Achene color closest to 191B. Attachment point of achene colored closest to 155C. Distal tip of achene colored 163C at the floral scar.

### COMPARISONS TO RELATED TURPENTINE BUSHES

The inventor knows no other patented Ericameria laricifolia cultivars. Two existing unpatented cultivars have been available in the nursery trade. ‘Desert Mountain’ is a seed based cultivar from a wild population in North Scottsdale. The plants are quite variable as this is an open pollinated population of plants. ‘Desert Mountain’ has been widely used for revegetation in the Phoenix area. In general these plants have a feathery form growing to 2-3 feet tall×2-3 feet wide. This cultivar seems to have gone out of production, as the original grower no longer lists it either on its availability or grow lists. Cultivar ‘Aguirre’ is a clonal selection from New Mexico selected for showy flowers and rich green foliage. This cultivar is listed as growing to 2-3 feet tall and wide. A third cultivar called ‘Texas Canyon’ can be found listed on internet information sites, but no nurseries can be found that produce or sell it.

Compared to ‘Desert Mountain’, Ericameria ‘Butterbloom’ is more uniform, dense and even in growth habit. Bushes of ‘Butterbloom’ are rounded, compared to the feathery form of ‘Desert Mountain’.

Compared to ‘Aguirre’, ‘Butterbloom’ is more uniform, dense and less open in growth form. Compared to the other cultivar descriptions, ‘Butterbloom’ is larger and less tall than wide at 3 feet tall×5 feet wide in the 4 year old individual examined. It should be noted that the phenotype of this species is quite plastic in response to both light and available water, such that the existing cultivars might reach sizes similar to ‘Butterbloom’ under favorable conditions.

I claim:

1. A new and distinct Ericameria laricifolia plant substantially as described and illustrated herein.
